(19) United States
(12) Patent Application Publication
(10) Pub. No.: US 2006/0178633 A1
(43) Pub. Date: Aug. 10, 2006

(54) CHASSIS FOR FLUID DELIVERY DEVICE

(75) Inventors: John T. Garibotto, Marblehead, MA (US); Steven DiIanni, Danvers, MA (US); David Zeller, Medford, MA (US); Brian James, Reading, MA (US); Marc Anderson, Clinton, MA (US); Fred Finnemore, Jefferson, ME (US); David Clare, Danvers, MA (US); Jason Daigle, Medford, MA (US)

Correspondence Address: INSULET CORPORATION, 9 Oak Park Drive, Bedford, MA, 01730, US

(73) Assignee: INSULET CORPORATION, Bedford, MA (US)

(21) Appl. No.: 10/907,286

(22) Filed: Mar. 28, 2005

Related U.S. Application Data
(60) Provisional application No. 60/649,518, filed on Feb. 3, 2005.

(51) Int. Cl. A61M 37/00

(52) U.S. Cl. 604/155

(57) ABSTRACT

A chassis for a fluid delivery device provides mechanical and/or electrical connections between components of the fluid delivery device. The chassis includes a framework of structural members that mechanically interface components, such as a batteries, electrical contacts, fluid reservoirs, tubing, drive wheels, pivoting actuator components, sensors, control circuitry, alarms or indicators, sliding assemblies, springs, cams and latching members. The chassis may also include one or more electrically conductive paths along selected portions of the framework to provide electrical connections between the components.

### CROSS-REFERENCE TO RELATED APPLICATIONS

This application claims the benefit of U.S. Provisional Patent Application Ser. No. 60/649,518 entitled CHASSIS FOR FLUID DELIVERY DEVICE, which was filed on Feb. 3, 2005, and is incorporated by reference herein. This application is related to U.S. patent application Ser. No. ______ (Attorney Docket No. INSL-171) entitled CHASSIS FOR FLUID DELIVERY DEVICE, which is filed concurrently herewith, assigned to the assignee of the present application, and incorporated herein by reference.

### TECHNICAL FIELD

The present invention relates to fluid delivery devices and more particularly, to a chassis for providing mechanical and/or electrical connections between components of a fluid delivery device.

### BACKGROUND INFORMATION

Fluid delivery devices have numerous uses such as delivering a liquid medicine to a patient subcutaneously. In a patient with diabetes mellitus, for example, ambulatory infusion pumps have been used to deliver insulin to a patient. These ambulatory infusion pumps have the ability to offer sophisticated fluid delivery profiles including variable basal rates and bolus requirements. The ability to carefully control drug delivery can result in better efficacy of the drug and therapy and less toxicity to the patient.

Some existing ambulatory infusion pumps include a reservoir to contain the liquid medicine and use electromechanical pumping or metering technology to deliver the liquid medicine via tubing to a needle and/or soft cannula that is inserted subcutaneously into the patient. These existing devices allow control and programming via electromechanical buttons or switches located on the housing of the device. The devices include visual feedback via text or graphic screens and may include alert or warning lights and audio or vibration signals and alarms. Such devices are typically worn in a harness or pocket or strapped to the body of the patient.

Currently available ambulatory infusion devices are expensive, difficult to program and prepare for infusion, and tend to be bulky, heavy and very fragile. Preparing these devices for infusion can be difficult and require the patient to carry both the intended medication and various accessories. Many existing devices also require specialized care, maintenance, and cleaning to assure proper functionality and safety for their intended long-term use. Due to the complexity and high cost of existing devices many patients who would benefit from an ambulatory infusion pump are, nonetheless, using inferior forms of therapy.

Accordingly, there is a need for a fluid delivery device with a reduced size and complexity and that is relatively inexpensive to manufacture.

### BRIEF DESCRIPTION OF THE DRAWINGS

These and other features and advantages will be better understood by reading the following detailed description, taken together with the drawings wherein:

FIG. 1 is a top view of a chassis for use in a fluid delivery device, consistent with one embodiment of the present invention.

FIG. 2 is a front view of the chassis shown in FIG. 1.

FIG. 3 is a back view of the chassis shown in FIG. 1.

FIG. 4 is a side view of the chassis shown in FIG. 1.

FIG. 5 is a top perspective view of a chassis with an electrically conductive path, consistent with another embodiment of the present invention.

FIG. 6 is a bottom perspective view of the chassis shown in FIG. 5.

FIG. 7 is a perspective view of a first shot molding for the chassis, consistent with one embodiment of the present invention.

FIG. 8 is a perspective view of a second shot molding for the chassis, consistent with one embodiment of the present invention.

FIG. 9 is a top view of a fluid delivery device, consistent with one embodiment of the present invention.

FIG. 10 is a top perspective view of a chassis providing a mechanical interface between a fluid reservoir and a fluid driving mechanism, consistent with one embodiment of the present invention.

FIG. 11 is a bottom perspective view of a chassis providing a mechanical interface for an actuating mechanism for the fluid driving mechanism, consistent with one embodiment of the present invention.

FIG. 12 is a top perspective view of a chassis providing a mechanical interface for an insertion mechanism and sensors, consistent with one embodiment of the present invention.

FIG. 13 is a top perspective view of a chassis providing a mechanical interface to a circuit board, consistent with one embodiment of the present invention.

FIGS. 14 and 15 are top perspective views of a chassis mounted to a circuit board and mechanically interfacing an insertion mechanism, consistent with one embodiment of the present invention.

FIG. 16 is a bottom perspective view of a chassis showing one embodiment of a fluid passage mechanism receptacle in greater detail.

FIG. 17 is an external perspective view of a reservoir assembly consistent with one embodiment of the present invention.

FIG. 18 is an internal perspective view of the reservoir assembly of FIG. 17.

### DETAILED DESCRIPTION

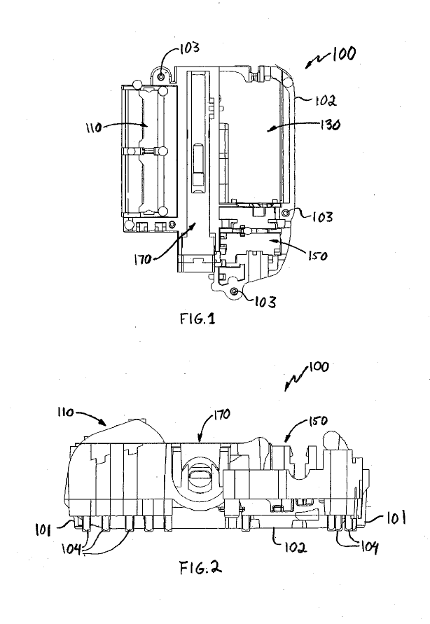

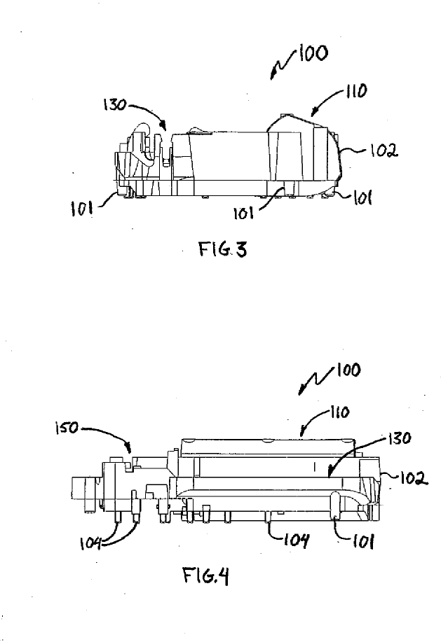

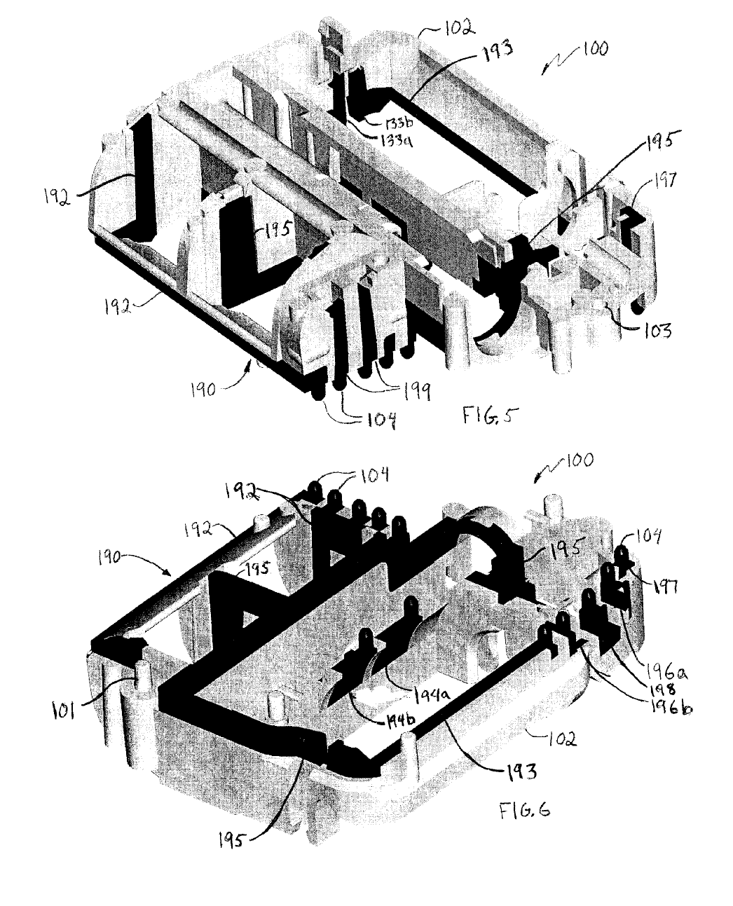

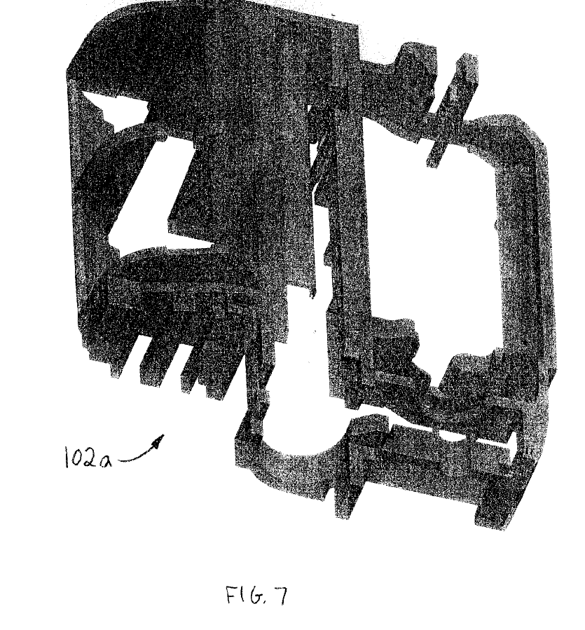

Referring to FIGS. 1-4, one embodiment of a chassis 100 for use with a fluid delivery device is shown and described. The chassis 100 provides mechanical and/or electrical connections between components of the fluid delivery device. In the exemplary embodiments shown and described herein, the chassis 100 is used in a fluid delivery device that subcutaneously delivers a fluid, such as a liquid medicine, to a person or an animal. Those skilled in the art will recognize that the chassis 100 may be used with other types of fluid delivery devices.

The chassis 100 includes a framework 102 of structural members that mechanically interface components of the fluid delivery device. As used herein, mechanical interface means to support components, engage components, attach to components, and/or position the components relative to other components. The components may include, but are not limited to, batteries, electrical contacts, fluid reservoirs, tubing, drive wheels, drive rods, pivoting actuator components, sensors, control circuitry, alarms or indicators, cams and sliding assemblies. Although specific configurations and shapes for the structural members of the framework 102 are shown, those skilled in the art will recognize that other configurations and shapes may be designed to interface with other types of components.

According to one embodiment of the chassis 100, the structural members of the framework 102 define receptacles for receiving, mechanically and/or electrically, components of the fluid delivery device. The chassis 100 may include a power source receptacle 110 for receiving a power source such as batteries, a reservoir receptacle 130 for receiving a fluid reservoir, a fluid driving mechanism receptacle 150 for receiving components of a fluid driving mechanism, and a fluid passage mechanism receptacle 170 for receiving components of a fluid passage mechanism. The structural members of the framework 102 may also include circuitry mounting members 104 for mounting to a control circuitry component, such as a printed circuit board. The structural members of the framework 102 may also include housing mounting posts 101 and/or post receptacles 103 for mechanically engaging an external housing 202 (see FIG. 9) that encloses the chassis 100. At final assembly, mounting posts 101 may be permanently attached to the housing with glue or other attachment means. Mounting posts 101 enable the chassis to provide additional structural integrity to the fluid delivery device by transmitting and distributing force over the housing.

Referring to FIGS. 5 and 6, the chassis 100 may also include one or more electrically conductive paths 190 (shown with dark shading) along portions of the structural members of the framework 102. The electrically conductive path(s) 190 may be formed along selected portions of the framework 102 to provide electrical connections between one or more of the components of the fluid delivery device, for example, between the power source, the control circuitry, the fluid driving mechanism, the sensors and other electronic components. The chassis 100 may also include an electrically conductive path (not shown) that forms an antenna for receiving signals, for example, transmitted from a remote control.

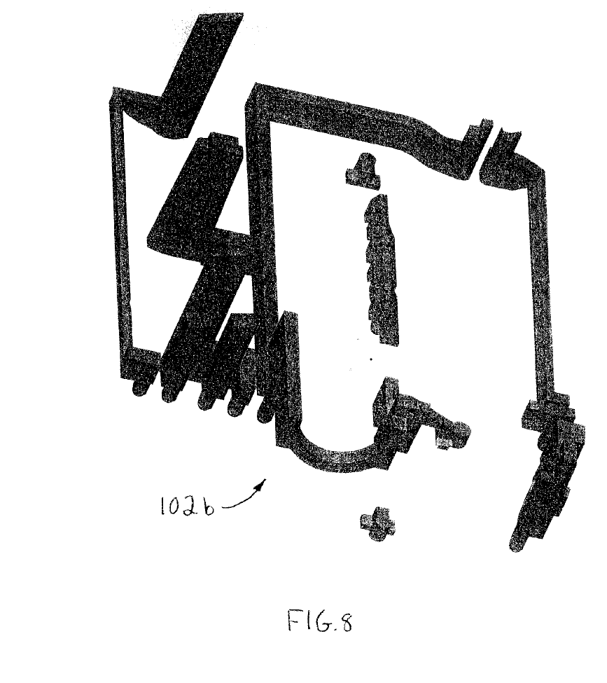

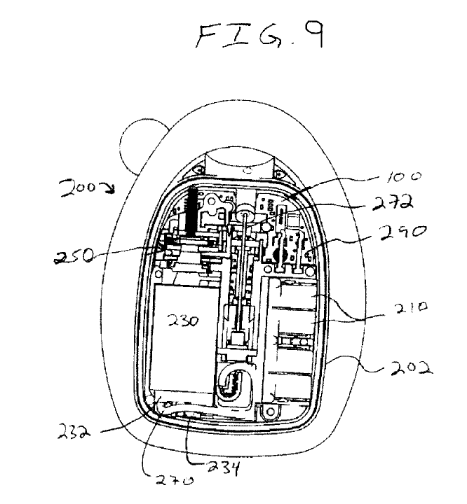

According to one method of making the chassis 100, the framework 102 may be formed by a two-shot molding process and then selectively plated to form the electrically conductive path(s) 190. Methods of selective plating, such as submerging the component in a substance that etches only the second shot material, for example, and then submerging the component in one or more baths that comprise the plating materials, are known to those of ordinary skill in the art. One example of a first framework section 102a formed by the first molding shot is shown in FIG. 7, and one example of a second framework section 102b formed by the second molding shot is shown in FIG. 8. The first shot or framework section 102a may be molded using a first plastic material such as polycarbonate, and the second shot or framework section 102b may be molded using a second plastic material such as ABS.

The second plastic material may be selected to be etched and thereby bond the conductive plating material(s), preferably nickel over copper. The plating will occur only on the molded plastic of the second framework section 102b formed by the second molding shot. The two-shot molding process therefore enables selective plating to form the conductive paths in desired locations along the chassis 100. Additional plating may also be used in selected areas to add structural integrity to the structural framework 102 of the chassis 100 or to achieve a desired surface finish. Multi-shot molding processes and plating processes known to those skilled in the art may be used. Those skilled in the art will also recognize that other methods may be used to form the framework 102 and to form the electrically conductive paths 190, such as insert molding or over-molding using one or more conductive elements, vapor deposition with masking, or vacuum deposition of a conductive material onto a molded plastic element.

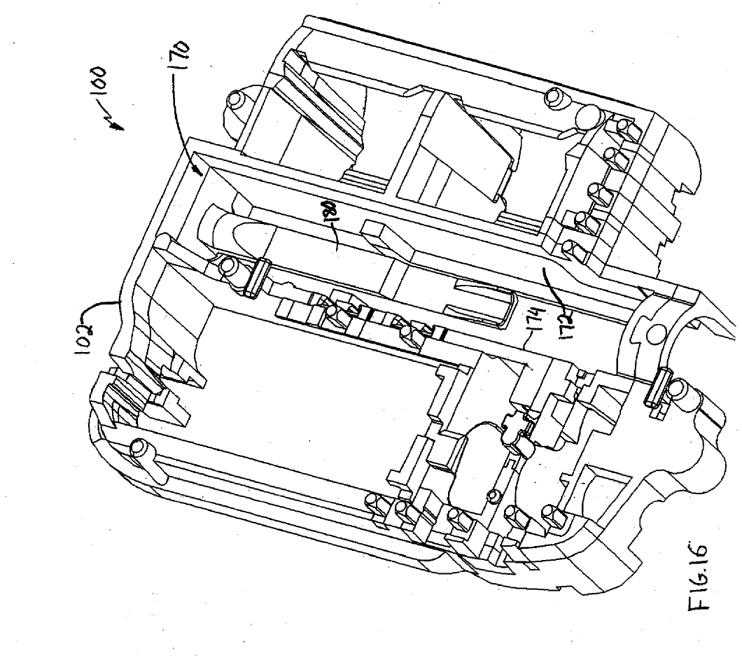

Referring to FIGS. 9-18, one embodiment of a fluid delivery device 200 is described in greater detail before describing the exemplary embodiment of the chassis 100 in greater detail. The components of the exemplary fluid delivery device 200 may include one or more batteries 210 for providing a power source, a fluid reservoir 230 for holding a fluid, a fluid driving mechanism 250 for driving the fluid out of the reservoir 230, a fluid passage mechanism 270 for receiving the fluid from the reservoir 230 and passing the fluid to a destination, and a control circuit board 290 with control circuitry for controlling the device. The fluid delivery device 200 may also include a housing 202 to enclose the components 210, 230, 250, 270, 290 and the chassis 100.

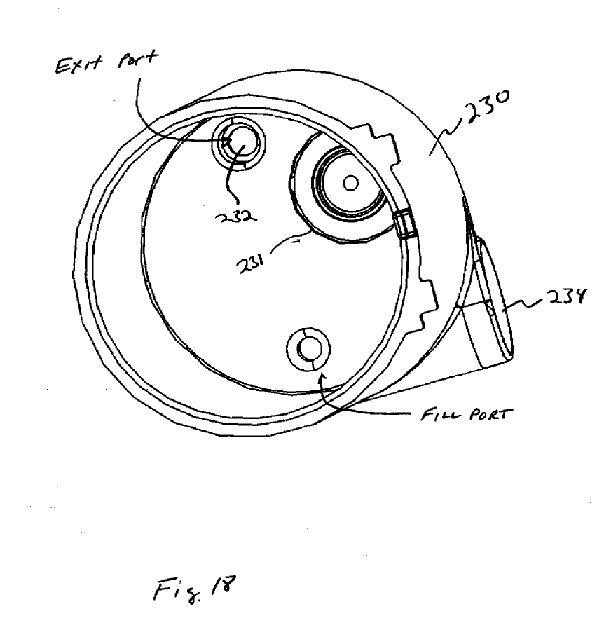

One embodiment of the reservoir 230 includes an outlet port 232 for allowing fluid to exit the reservoir 230 (FIGS. 17 and 18). The reservoir 230 may also include an inlet port 234 for allowing the reservoir 230 to be filled with fluid. The reservoir 230 may also include a contact element 233 received in an occlusion sensor membrane 231. A plunger 236 may be received in the reservoir 230 to force fluid out of the reservoir 230.

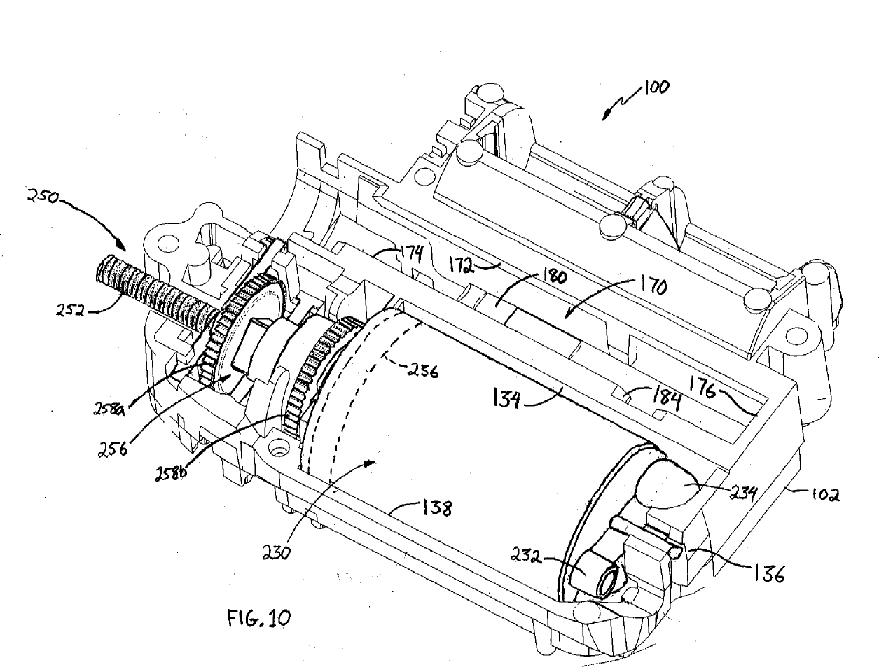

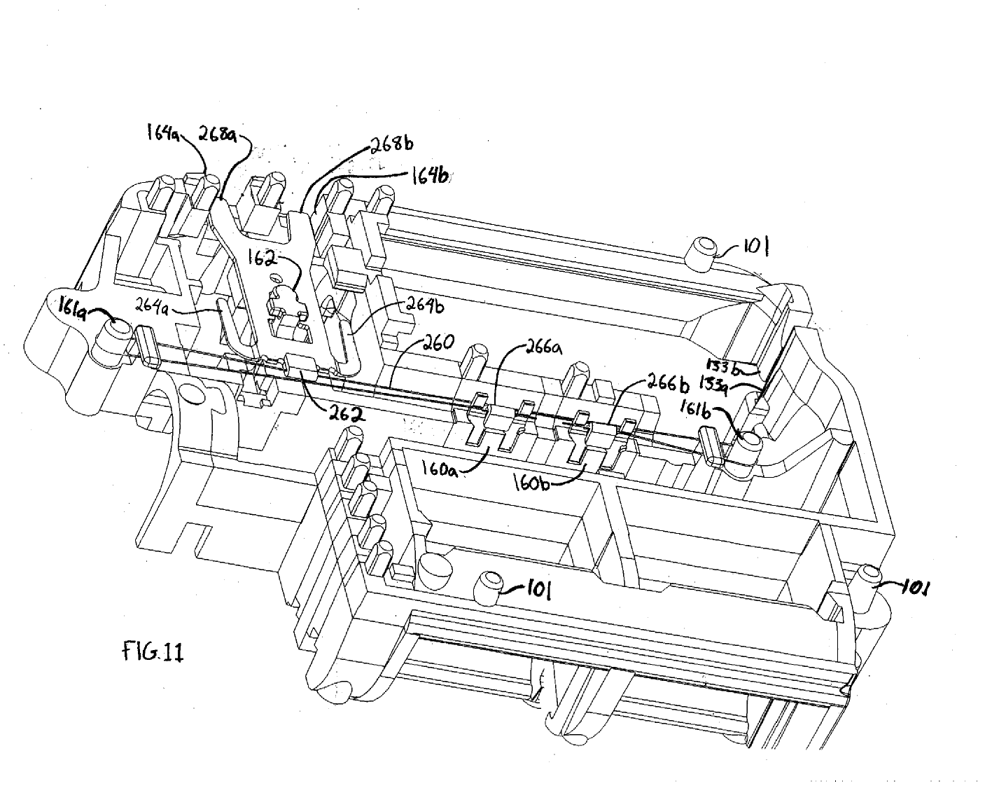

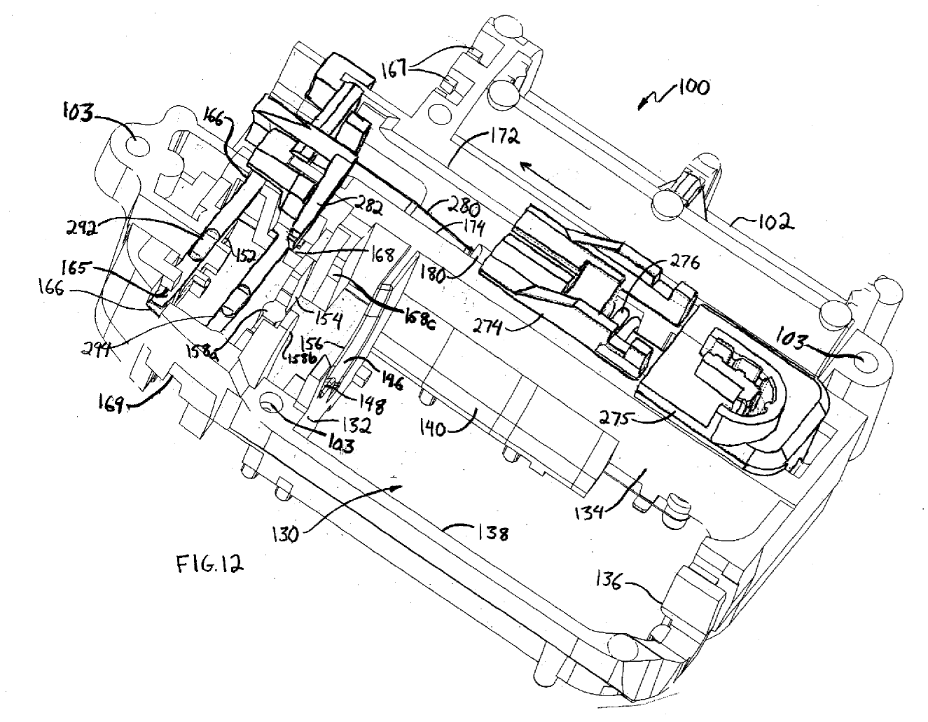

One embodiment of the fluid driving mechanism 250 includes a threaded drive rod 252 connected at one end to the plunger 236 received in the reservoir 230 (FIG. 10). A threaded drive wheel 256 threadably engages and imparts linear motion to the threaded drive rod 252 to advance the plunger 236 into the reservoir 230, thereby forcing fluid out of the reservoir 230. The drive wheel 256 may include ratchet wheel portions 258a, 258b.

An actuating mechanism for the drive wheel 256 may include a shape memory alloy (SMA) element 260 coupled to a pivotable drive engaging member 262 (FIG. 11). One embodiment of the SMA element 260 is a SMA wire connected at each end to terminations 266a, 266b. One embodiment of the drive engaging member 262 includes arms 264a, 264b for engaging the ratchet teeth on the ratchet wheels 258a, 258b and legs 268a, 268b for providing electrical contacts. A change in shape of the SMA element 260 causes the arms 264a, 264b of the drive engaging member 262 to engage ratchet teeth on wheels 258a, 258b, thereby rotating the drive wheel 256.

Alternative drive mechanisms and actuating mechanisms that may, for example, be accommodated in a chassis in accordance with the present invention are disclosed in U.S. Pat. Nos. 6,656,158 and 6,656,159 and U.S. patent application Ser. No. 10/704,291, all of which are hereby incorporated by reference.

One embodiment of the fluid passage mechanism 270 includes a transcutaneous access tool 272, such as a needle and/or soft cannula, which is capable of penetrating the skin of a patient and passing the fluid into the patient (FIG. 9). A fluid path such as tubing (not shown) may be used to fluidly couple the reservoir 230 to the transcutaneous access tool 272. The access tool 272 is mounted to an insertion mechanism, which may include sliding carriages 274, 275, one or more springs 276, and a release member 280 (FIG. 12). The sliding carriages 274, 275 may be held in a first retracted position until the release member 280 causes the sliding carriages 274, 275 to be released and the spring(s) 276 drive the sliding carriages 274, 275 in the direction of the arrow into an insertion position. The drive mechanism 250 may be used to trigger the release member 280 by engaging an arm 282 of the release member 280. In one embodiment, the sliding carriages 274, 275 first insert a needle and a soft cannula and then the sliding carriage 275 withdraws the needle leaving the soft cannula in place. Various transcutaneous access devices and systems that may be accommodated in a chassis in accordance with the present invention are disclosed in, for example, U.S. Pat. No. 6,656,159 and U.S. patent application Ser. Nos. 10/128,206, 10/195,745, 10/260,192, 10/261,003, all of which are hereby incorporated by reference.

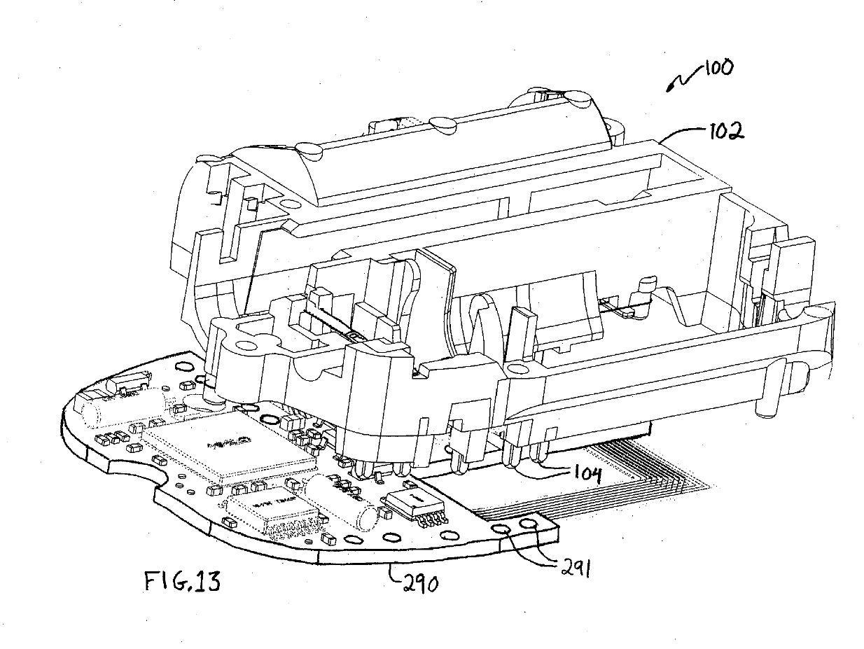

One embodiment of the control circuit board 290 includes control circuitry for controlling operation of the fluid delivery device 200, for example, by controlling the actuating mechanism for the fluid driving mechanism 250 (FIG. 13). For example, the control circuitry may initially actuate the fluid driving mechanism 250 to cause the transcutaneous access tool 272 to be inserted. The control circuitry may then actuate the fluid driving mechanism 250 to precisely control the delivery of fluid. The control circuit board 290 may also include control circuitry for monitoring operation of the fluid delivery device 200, for example, by receiving signals from one or more components, such as the actuating mechanism. The control circuit board 290 may also include communications circuitry for communicating with a remote controller.

The fluid delivery device 200 may also include one or more sensors that provide signals to the control circuitry to monitor and control the fluid delivery device 200. For example, a fill sensor 292 may be used to indicate the amount of fluid in the reservoir 230 and a safety sensor 294 may be used to indicate proper operation of the fluid driving mechanism 250 (FIG. 12). The fluid delivery device 200 may also include a signaling indicator 298 such as a piezo or other audible alarm, a vibration element and/or a visual indicator (FIG. 9). The signaling indicator or alarm may be mounted to the housing 202. The control circuitry may also include an antenna either on the circuit board 290, on a separate structure, or on the chassis 100 for receiving signals, for example, from a remote control device.

The exemplary embodiment of the chassis 100 is now described in greater detail in connection with the components of the exemplary fluid delivery device 200 described above.

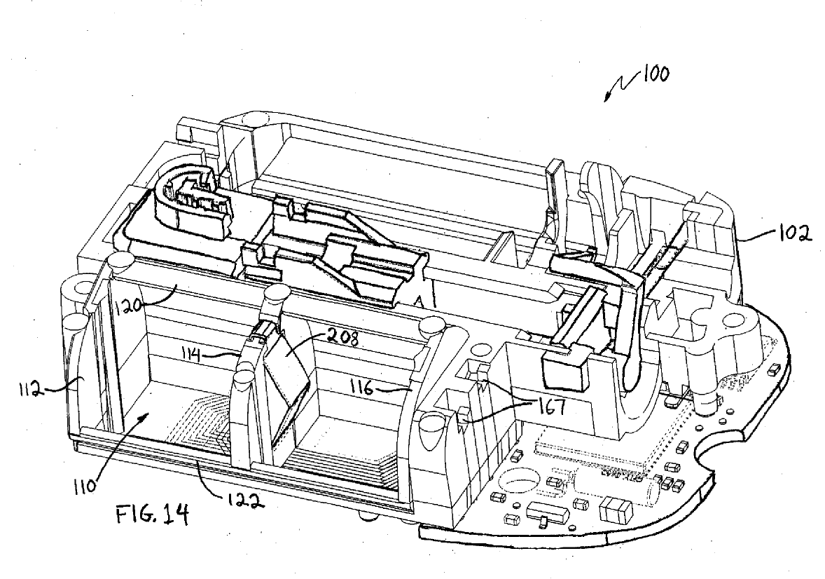

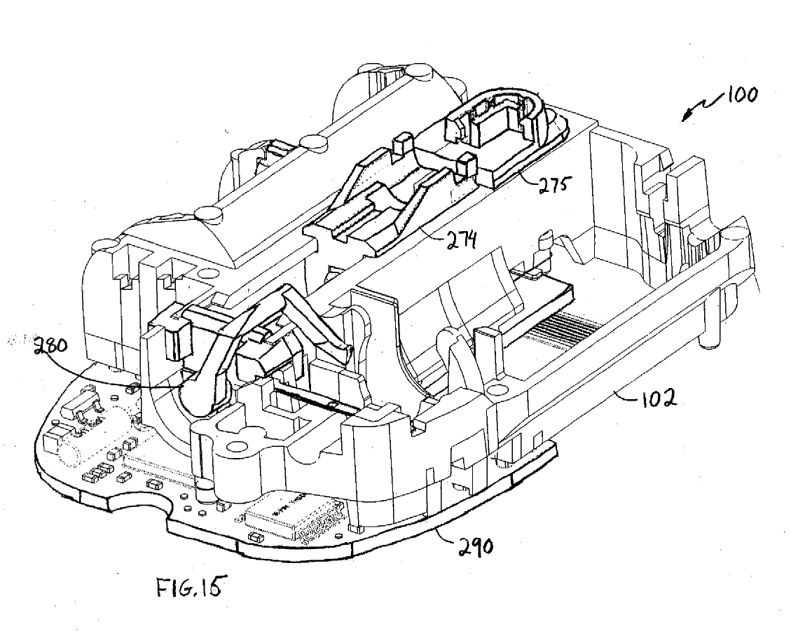

The structural members defining the power source receptacle 110 may include side walls 112, 114, 116 and top and bottom walls 120, 122 configured to receive and support batteries (FIG. 14). One or more power conductive paths 192 and/or a common ground conductive path 195 (FIGS. 5 and 6) may extend along one or more of the power source receptacle walls 112, 114, 116, 120, 122 to provide a power connection to the control circuitry. At least one wall 114 of the power source receptacle 110 may also be configured to receive a battery contact 208 in electrical connection with the power conductive path 192 on that wall 114.

The structural members defining the reservoir receptacle 130 may include side walls 132, 134, 136, 138 and a support member 140 configured to receive and support the fluid reservoir 230 (FIGS. 10 and 12). The side walls 132, 134, 136, 138 and support member 140 are located in the framework 102 to position the reservoir 230 relative to the fluid driving mechanism 250. One or more of the side walls may be configured to engage a portion of the reservoir 230 to hold the reservoir 230 in place. A portion of the structural members defining reservoir receptacle 130 may include electrical contacts 133a and 133b (FIGS. 5 and 11). Electrical contacts 133a and 133b are adapted to contact occlusion sensor contact 233 (FIGS. 17 and 18), which is preferably an electrically conductive spherical contact element 233 adapted to complete a circuit between contacts 133a and 133b, when biased into engagement with contacts 133a and 133b. In a relaxed state, the occlusion sensor membrane 231 on the reservoir 230 holds the contact element 233 out of contact with contacts 133a and 133b. At a predetermined pressure selected to coincide with an occlusion condition, the membrane 231 and the contact element 233 is displaced into contact with the contacts 133a and 133b. The contact 133a may be electrically connected to the common ground conductive path 195 and the contact 133b may be connected to an occlusion sensor conductive path 193.

The structural members defining the fluid driving mechanism receptacle 150 may include walls 152, 154, 156 configured to receive the drive wheel 256 (FIG. 12). The walls 152, 154, 156 are located in the framework 102 to position the fluid driving mechanism 250 relative to the reservoir 230 and drive engaging member 262, which allows the drive rod 252 to advance the plunger 236 into the reservoir 230. At least one wall 154 may provide rotating bearing surfaces and thrust surfaces 158a for the drive wheel 256 and at least one of the walls 152 may support the threaded drive rod 252. Additional bearing surfaces 158b, 158c on the chassis 100 may also contain and/or support the drive wheel 256. The framework 102 may also provide a guide surface 146 and/or a camming surface 148 for engaging a tilt nut used to couple the drive rod to the drive wheel.

The structural members of the framework 102 may also provide actuator attachment points 160a, 160b for the SMA element 260 and a pivot point 162 for the pivotable drive engaging member 262 (FIG. 11). In one embodiment, the actuator attachment points 160a, 160b receive and attach to the terminations 266a, 266b at each end of the SMA element 260. The chassis 100 may also include posts 161a, 161b or other supporting structures for the SMA element 260. The pivot point 162 is located on the framework 102 such that the pivotable drive engaging member 262 engages the drive wheel 256. One or more actuator conductive paths 194a, 194b (FIGS. 5 and 6) may extend from the attachment points 160a, 160b and the common ground conductive path 195 may extend from the pivot point 162. The framework 102 also provides contact points 164a and 164d that contact the legs 268a, 268b of the drive engaging member 262. Actuator conductor paths 196a, 196b (FIGS. 5 and 6) also extend from the contact points 164a and 164b. The actuator conductive paths 194a, 194b 196a, 196b and the common ground conductive path 195 provide an electrical connection between the actuating mechanism and the control circuitry.

The structural members of the framework 102 may also include sensor supports or electromechanical attachment points 166, 168 for supporting or mounting sensors such as the fill sensor 292 and the safety sensor 294 and sensor contact points 165, 169 for contacting the sensor 292, 294, respectively (FIG. 12). The chassis 100 may also provide one or more electromechanical attachment points 167 for electrical contacts for the signaling indicator 298. The common ground conductive path 195 may extend from the sensor supports 166, 168, and one or more sensor conductor paths 197, 198 (FIGS. 5 and 6) may extend from the sensor contact points 165, 169, respectively, to provide an electrical connection between the sensors 292, 294 and the control circuitry. One or more indicator conductive paths 199 (FIGS. 5 and 6) extend from the attachment point(s) 167 and provide an electrical connection between the indicator 298 and the control circuitry.

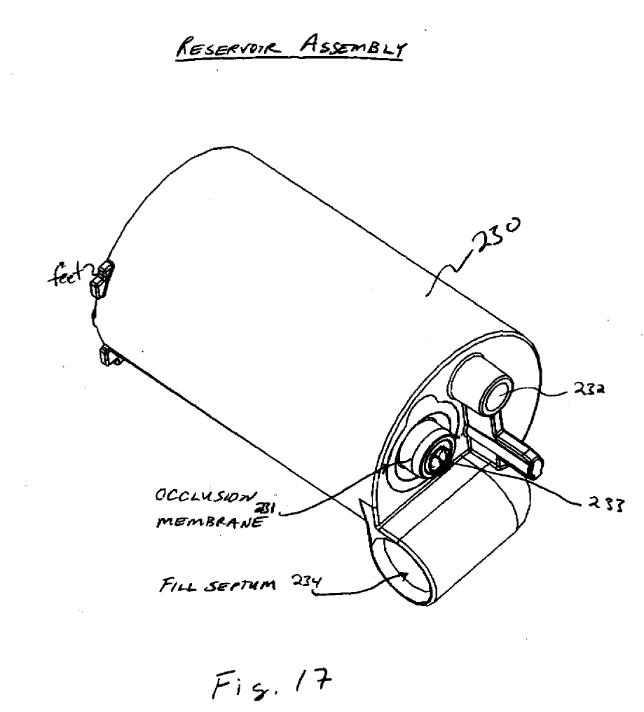

The structural members defining the fluid passage mechanism receptacle 170 may include side walls 172, 174 and rear wall 176 (FIGS. 10 and 16). The side walls 172, 174 are located in the framework 102 to receive and allow sliding movement of the sliding carriages 274, 275 relative to the chassis 100 (FIG. 12). A latch arm 180 may extend from the rear wall 176 to engage the sliding carriages 274, 275 on a latch surface of the arm 180 and hold the sliding carriages 274, 275 in the first retracted position. In a preferred embodiment, the latch arm 180 is biased into engagement with the carriages 274, 275 by the release member 280. The release member 280 may be mounted at the end of the side walls 172, 174 and positioned such that the release member 280 can engage the drive wheel 256 and the latch arm 180. Initial actuation of the drive wheel 256 causes the release member 280 to engage and move the latch arm 180, which releases the sliding carriages 274, 275 from the first retracted position. At least one wall 174 may include a catch surface 184 for engaging the sliding carriage 275 in a retracted position.

One embodiment of the mounting members 104 may include one or more mounting pegs that are inserted into holes 291 in the circuit board 290 (FIG. 13) or in an attachment to the circuit board (not shown). The pegs may have a square or other shape that mechanically engages the circuit board, for example, in a friction fit, press fit, compliant fit. The pegs may be further secured to the board by other means such as solder, heat stake or ultrasonic stake. Alternatively, the chassis may have female elements that mate with corresponding male elements on the circuit board or an attachment thereto. The power conductive path(s) 192, actuator conductive paths 194a, 194b, 196a, 196b, common ground conductive path 195, sensor conductive path(s) 197, 198 and signaling indicator conductive path(s) 199 extend along portions of the framework 102 to the mounting members 104 (FIGS. 5 and 6) or other contact points with the circuit board. The mounting members 104 are positioned on the framework 102 to electrically connect the conductive paths 192-199 to the appropriate locations on the circuit board 290. One or more of the mounting members 104 may also electrically connect the circuit board 290 to an antenna conductive path (not shown) connected to an antenna formed on the chassis 100.

Consistent with one embodiment of the invention, the fluid delivery device includes a fluid reservoir configured to hold a fluid and a fluid passage mechanism fluidly coupled to the fluid reservoir. A fluid driving mechanism forces the fluid from the fluid reservoir and through the fluid passage mechanism. Control circuitry controls and monitors the operation of the fluid delivery device. A chassis including a framework of structural members mechanically interfaces the fluid reservoir, the fluid passage mechanism, the fluid driving mechanism, and the control circuitry.

Consistent with another embodiment of the present invention, a fluid delivery device includes fluid delivery components and a chassis including a framework of structural members for receiving and mechanically interfacing at least some of the fluid delivery device components. The chassis also includes at least one electrically conductive path along a portion of the structural members for providing electrical connections between at least some of the fluid delivery device components.

Consistent with a further embodiment of the present invention, a chassis includes a power source receptacle configured to receive a power source, a fluid reservoir receptacle configured to receive a fluid reservoir, a fluid passage mechanism receptacle configured to receive a fluid passage mechanism, and a fluid driving mechanism receptacle configured to receive a fluid driving mechanism. The fluid reservoir receptacle, fluid passage mechanism receptacle and fluid driving mechanism receptacle are configured to mechanically interface the fluid reservoir, fluid passage mechanism and fluid driving mechanism with respect to each other.

Consistent with yet another embodiment of the present invention, a chassis includes a framework of structural members for receiving and mechanically interfacing components of the fluid delivery device, and at least one electrically conductive path along a portion of the structural members for providing electrical connections between components of the fluid delivery device.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

What is claimed is:

1. A fluid delivery device comprising:
a fluid reservoir configured to hold a fluid;
a fluid passage mechanism fluidly coupled to said fluid reservoir;
a fluid driving mechanism configured to force said fluid from said fluid reservoir and through said fluid passage mechanism;
control circuitry for controlling and monitoring operation of said fluid delivery device; and
a chassis comprising a framework of structural members mechanically interfacing said fluid reservoir, said fluid passage mechanism, said fluid driving mechanism, and said control circuitry.

2. The fluid delivery device of claim 1 wherein said chassis provides electrical connections between at least a power source, said control circuitry and said fluid driving mechanism.

3. The fluid delivery device of claim 1 wherein said chassis is multi-shot molded.

4. The fluid delivery device of claim 2 wherein said chassis is multi-shot molded and selectively plated with conductive material to form electrically conductive paths to provide at least one of said electrical connections.

5. The fluid delivery device of claim 1 wherein said chassis comprises:
a power source receptacle configured to receive a power source;
a fluid reservoir receptacle configured to receive said fluid reservoir;
a fluid passage mechanism receptacle configured to receive said fluid passage mechanism; and
a fluid driving mechanism receptacle configured to receive said fluid driving mechanism and to provide a mechanical connection between said fluid driving mechanism and said fluid passage mechanism.

6. The fluid delivery device of claim 1 wherein said fluid passage mechanism comprises a transcutaneous access tool fluidly coupled to said reservoir and an insertion mechanism configured to move said transcutaneous access tool from a retracted position to an insertion position, wherein said chassis includes at least one of a catch surface and a latch surface configured to engage said insertion mechanism holding said transcutaneous access tool in a retracted position.

7. The fluid delivery device of claim 6 wherein said chassis supports a release member between said fluid driving mechanism and said insertion mechanism such that actuation of said fluid driving mechanism causes said release member to release said insertion mechanism from said retracted position.

8. The fluid delivery device of claim 1 wherein said fluid reservoir includes a plunger for forcing fluid out of said reservoir, wherein said fluid driving mechanism includes a threaded drive rod coupled to said plunger and a threaded drive wheel for imparting linear motion to said threaded drive rod to advance said plunger, and wherein said chassis provides at least one bearing surface for said drive wheel.

9. The fluid delivery device of claim 8 wherein said fluid driving mechanism further comprising an actuating mechanism mechanically and electrically connected to said chassis for actuating said drive wheel.

10. The fluid delivery device of claim 9 wherein said actuating mechanism comprises a shape memory alloy element electrically and mechanically coupled to said chassis and a pivotable drive engaging member coupled to said shape memory alloy and pivotably coupled to said chassis for pivoting to engage said drive wheel.

11. The fluid delivery device of claim 1 wherein said control circuitry comprises a circuit board, and wherein said chassis comprises mounting elements, said mounting elements providing electrical contacts for providing electrical connections between said circuit board and electrically conductive paths on said chassis when said circuit board is coupled to said chassis with said mounting elements.

12. The fluid delivery device of claim 1 further comprising at least one sensor mechanically interfaced to said chassis, wherein said chassis provides electrical connections between at least a power source, said at least one sensor, and said control circuitry.

13. The fluid delivery device of claim 1 further comprising a signaling indicator, wherein said chassis provides electrical connections between at least a power source, said signaling indicator and said control circuitry.

14. The fluid delivery device of claim 1 wherein said chassis includes electrically conductive paths forming an antenna, and wherein said chassis provides an electrical connection between said antenna and said control circuitry.

15. The fluid delivery device of claim 1 further comprising a housing for housing said chassis, said fluid reservoir, said fluid passage mechanism and said fluid driving mechanism.

16. A fluid delivery device comprising:
fluid delivery components; and
a chassis comprising a framework of structural members for receiving and mechanically interfacing at least some of said fluid delivery device components, and at least one electrically conductive path along a portion of said structural members for providing electrical connections between at least some of said fluid delivery device components.

17. The fluid delivery device of claim 16 wherein said structural members include at least one of a bearing surface and a camming surface for at least one of said fluid delivery components.

18. The fluid delivery device of claim 16 wherein said structural members include at least one of a catch surface and a latch surface for at least one of said fluid delivery components.

19. The fluid delivery device of claim 16 wherein said chassis includes electromechanical attachment points providing both mechanical connections and electrical connections to at least some of said fluid delivery components.

20. The fluid delivery device of claim 16 wherein said fluid delivery components include control circuitry mechanically and electrically connected to electromechanical attachment points on said chassis and a fluid driving mechanism including components mechanically and electrically connected to electromechanical attachment points on said chassis, wherein said electrically conductive path extends between said attachment points.

21. The fluid delivery device of claim 16 wherein said chassis is multi-shot molded and selectively plated with conductive material to form said electrically conductive path.

22. The fluid delivery device of claim 16 wherein said chassis is formed by over-molding plastic over one or more conductive elements.

23. The fluid delivery device of claim 16 wherein said chassis is formed by selective vacuum deposition of a conductive material onto a molded plastic element.

24. A chassis for a fluid delivery device, said chassis comprising:
a power source receptacle configured to receive a power source;
a fluid reservoir receptacle configured to receive a fluid reservoir;
a fluid passage mechanism receptacle configured to receive a fluid passage mechanism; and
a fluid driving mechanism receptacle configured to receive a fluid driving mechanism, wherein said fluid reservoir receptacle, said fluid passage mechanism receptacle and said fluid driving mechanism receptacle are configured to mechanically interface said fluid reservoir, said fluid passage mechanism and said fluid driving mechanism with respect to each other.

25. The chassis of claim 24 further comprising a framework of structural members forming said receptacles.

26. The chassis of claim 25 wherein said framework comprises mounting pegs configured to be mounted to a control circuit board.

27. The chassis of claim 25 wherein at least one of said structural members includes a latch surface configured to engage a component of said fluid passage mechanism.

28. The chassis of claim 25 wherein at least one of said structural members includes a bearing surface configured to engage a component of said fluid driving mechanism.

29. The chassis of claim 24 further comprising an electrically conductive path extending from at least said power source receptacle and said fluid driving mechanism receptacle.

30. The chassis of claim 29 further comprising electromechanical attachment points for mechanically and electrically connecting to control circuitry, wherein said electrically conductive path extends from at least said power source receptacle and said fluid driving mechanism receptacle to said electromechanical attachment points.

31. A chassis for a fluid delivery device, said chassis comprising:
a framework of structural members for receiving and mechanically interfacing components of said fluid delivery device; and
at least one electrically conductive path along a portion of said structural members for providing electrical connections between components of said fluid delivery device.

32. The chassis of claim 31 wherein said framework is selectively plated with conductive material to form said electrically conductive path.

33. The chassis of claim 31 wherein said framework is two-shot molded and selectively plated with conductive material to form said electrically conductive path.

34. The chassis of claim 31 wherein said framework defines receptacles for receiving said components.

35. The chassis of claim 31 wherein said structural members include mounting pegs for mounting to a circuit board.

36. The chassis of claim 31 wherein said structural members include electromechanical attachment points configured to mechanically engage and electrically connect to components of said fluid delivery device, wherein said electrically conductive path extends between said electromechanical attachment points.

37. The chassis of claim 31 wherein said structural members include actuator mounting points configured to mechanically mount an actuating mechanism, and wherein said electrically conductive path extends to said actuator mounting points.

38. The chassis of claim 31 wherein said structural members include at least one bearing surface configured to receive at least one rotating component.

39. The chassis of claim 31 wherein said structural members include at least one latch surface configured to engage at least one moving component.

40. A method of assembling a fluid delivery device, said method comprising:
providing a chassis comprising a framework of structural members forming receptacles for components of said fluid delivery device;
placing said components into said receptacles such that said structural members mechanically interface said components; and
enclosing said chassis and said components in a housing.

41. The method of claim 40 further comprising electrically connecting at least some of said components to electrically conductive paths on said chassis.
